(19) United States
(12) Patent Application Publication
(10) Pub. No.: US 2026/0204365 A1
(43) Pub. Date: Jul. 16, 2026

(54) DEVICE AND METHOD FOR IMPRINTING AN ACTIVE EFFECT OF AT LEAST ONE REFERENCE PRODUCT INTO A TREATMENT PRODUCT

(71) Applicant: AEQUILLIVING INC LIMITED, London, Greater London (GB)

(72) Inventors: Frédéric Roscop, London (GB); Marie-Valérie Moreno, Neuveville-sur-Saône (FR)

(73) Assignee: AEQUILLIVING INC LIMITED, London, Greater London (GB)

(21) Appl. No.: 19/141,206

(22) PCT Filed: Dec. 20, 2023
(86) PCT No.: PCT/EP2023/087145

§ 371 Date: Jun. 19, 2025

(30) Foreign Application Priority Data
Dec. 20, 2022 (FR) FR2214017

(51) Int. Cl. G16C 20/90; C02F 1/48; C02F 103/02; G06F 17/14; G11C 13/00

(52) U.S. Cl. CPC G16C 20/90; C02F 1/484; G06F 17/142; G11C 13/0014; C02F 2103/02; C02F 2201/48

(57) ABSTRACT

THE method for imprinting an active effect of at least one reference product into a treatment PRODUCT INCLUDES a second imprinting phase (E2) comprising: —reading (E21) the associated digital signature of the at least one reference product from a database; —forming (E23) an electrical imprinting signal from the digital signature of the reference product; —imprinting (E24) the active effect into a neutral solution HAVING at least one salt, by passing the electrical imprinting signal through one or more electrical wires of a plate of an imprinting device on which the neutral solution is positioned, for a predetermined imprinting duration, so as to imprint the active effect of the at least one reference product into the neutral solution and thus transform it into a treatment product.

The invention relates to a method for capturing a digital signature of a reference product with a view to preparing an active product. The invention also relates to a method for imprinting an active effect of at least one reference product into a treatment product. The invention furthermore relates to a method for manufacturing a treatment product. The invention also relates to a capture machine and/or a machine for imprinting an active effect. The invention also relates to a computer program implementing one of the abovementioned methods. The invention relates, lastly, to a recording medium on which such a program is recorded.

Research has been conducted on the ability of a medium, such as water, a polymer substrate (such as glass or some plastics) to store information, in particular via electromagnetic fields emitted by living elements and minerals, including stones. Continuing on from this work, technical solutions have been sought for capturing such electromagnetic fields and transferring them to a medium, in particular imprinting them into an aqueous solution, in order to obtain an active product.

However, the technical solutions stemming from this work have drawbacks. In particular, the operation of existing capture and imprinting systems may be disturbed by the Earth's electromagnetic field or electromagnetic fields emitted in the environment of the capture systems.

The aim of the invention is to provide a capturing and imprinting device and method that improve the capturing and imprinting devices and methods known from the prior art. In particular, the invention makes it possible to produce a device and a method that are simple and reliable.

To this end, the invention relates to a method for structuring a medium containing water molecules, comprising a step of emitting electromagnetic waves into said medium.

The invention also relates to a method for imprinting an active effect of at least one reference product into a treatment product, characterized in that it comprises a second imprinting phase comprising the following steps:
Reading the associated digital signature of the at least one reference product from a database;
Constructing an electrical imprinting signal based on the digital signature of the reference product;
Imprinting the active effect into a neutral solution comprising at least one salt, by passing the electrical imprinting signal through one or more electrical wires of a plate of an imprinting device on which the neutral solution is positioned, for a predetermined imprinting duration, so as to imprint the active effect of the at least one reference product into the neutral solution and thus transform it into a treatment product.

In one embodiment, the medium comprises at least one salt selected from magnesium chloride and sodium chloride, and the water molecules in the medium structure themselves around crystals of the at least one salt.

In one embodiment, the structuring method is for the purpose of cooling the medium containing the water molecules.

In one embodiment, the electromagnetic waves are emitted over a frequency range between 120 and 30000 Hz, or even between 1000 and 5000 Hz.

In one embodiment, the electromagnetic waves are generated by an electrical structuring signal having a maximum current strength of between 8 and 96 μA, or even between 10 and 20 μA.

The invention furthermore relates to a method for capturing a digital signature of a reference product with a view to preparing an active product comprising the active effect of said reference product.

The capture method comprises the following steps:
Recording an electrical signal measured between at least two measuring electrodes of a capture device comprising a plate on which the reference product is positioned, the plate comprising one or more electrical wires capable of generating a virtual coil on a volume containing the reference product;
Processing the recorded electrical signal to form a digital signature comprising a frequency spectrum of the recorded electrical signal;
Storing the digital signature in a database.

The invention furthermore relates to a method for imprinting an active effect of at least one reference product into a treatment product.

The capture method comprises the following steps:
Reading the digital signature of the at least one reference product from a database;
Constructing an electrical imprinting signal based on the digital signature of the reference product;
Imprinting the active effect into a neutral solution comprising at least one salt, by passing the electrical imprinting signal through one or more electrical wires of a plate of an imprinting device on which the neutral solution is positioned, for a predetermined imprinting duration, so as to imprint the active effect of the at least one reference product into the neutral solution and thus transform it into a treatment product.

In one embodiment, at least two wires of the set of electrical wires are in parallel. As an alternative, at least two wires of the set of electrical wires are arranged in a spiral.

In one embodiment, the imprinting method furthermore comprises a step of determining imprinting parameters, comprising a chosen imprinting duration, and the duration for which the step of imprinting the active effect is carried out is equal to the chosen imprinting duration.

In one embodiment, the step of constructing an electrical imprinting signal based on the digital signature of the reference product comprises:
obtaining a first electrical signal by applying an inverse Fourier transform to the at least one digital signature of the reference product, and optionally
generating a set of harmonics of the first electrical signal, and then adding the set of harmonics to the first electrical signal, the set of harmonics comprising second and/or third and/or fourth and/or fifth harmonics.

The invention furthermore relates to a method for manufacturing a treatment product.

The manufacturing method comprises a first phase of capturing at least one digital signature of a reference product by implementing a capture method according to the invention, and
a second phase of imprinting at least one active effect into a neutral solution based on said digital signature by implementing the imprinting method according to the invention.

In one embodiment, the manufacturing method comprises an additional phase of structuring a medium containing water molecules by
implementing a structuring method according to the invention, and the additional phase is carried out before and/or during the first capture phase in order to structure the reference solution, and/or
the additional phase is carried out before and/or during the second imprinting phase in order to structure the treatment product.

The invention furthermore relates to a method for cooling a medium containing water molecules, comprising a step of structuring the medium by way of a structuring method according to the invention.

The invention furthermore relates to a device for imprinting an active effect, characterized in that it comprises:
A treatment plate comprising one or more electrical wires;
Access to a database comprising the storage of a digital signature of at least one reference product;
A control unit comprising hardware means and software means, configured to implement an imprinting method as described above, and/or a manufacturing method as described above, the control unit comprising a link firstly to said database for storing and/or reading at least one digital signature in/from said database and secondly to the one or more electrical wires of the plate for generating and/or measuring an electrical signal;
A human-machine interface for starting and/or interrupting the structuring and/or the capturing and/or the imprinting and/or the manufacturing and/or the cooling and for inputting structuring and/or capturing and/or imprinting and/or manufacturing and/or cooling parameters.

The invention also relates to a capture device and/or a device for imprinting an active effect or a structuring device, comprising:
A treatment plate comprising one or more electrical wires;
Access to a database comprising the storage of a digital signature of at least one reference product;
A control unit comprising hardware means and software means, configured to implement a structuring method according to the invention and/or a capture method according to the invention and/or an imprinting method according to the invention and/or a manufacturing method according to the invention and/or a cooling method according to the invention, the control unit comprising a link firstly to said local or remote database for storing and/or reading at least one digital signature in/from said database and secondly to the one or more electrical wires of the plate for generating and/or measuring an electrical signal;
A human-machine interface for starting and/or interrupting the structuring and/or the capturing and/or the imprinting and/or the manufacturing and/or the cooling and for inputting structuring and/or capturing and/or imprinting and/or manufacturing and/or cooling parameters.

In one embodiment, the capture device and/or the device for imprinting an active effect and/or the structuring device according to the invention comprises at least a first and second measuring and/or injection electrode, and one or more wires are connected to the first and second measuring and/or injection electrode in an electrical circuit, such that a first end of each of said one or more wires is connected to the first measuring and/or injection electrode and a second end of each of said one or more wires is connected to the second measuring and/or injection electrode.

In one embodiment, the plate comprises a set of electrical wires, and the wires of the set of electrical wires are or are not equidistant from one another. The wires may be substantially rectilinear and parallel. As a variant, it comprises one or more wires in the shape of a spiral.

In one embodiment, the plate comprises a plurality of wires and a first and second zone, such that a first distance measured between each pair of contiguous wires in the first zone differs from a second distance measured between each pair of contiguous wires in the second zone.

In one embodiment, wires in the at least one or at least two zones of the plate are capable of generating equivalent electric fields for imprinting the same active effect in various volumes of a neutral solution.

In one embodiment, the capture parameters comprise:
a selection of at least one zone of the plate used for the capture, referred to as capture zone, and/or
a capture mode, which may be a first capture mode referred to as capture mode “without structuring”, or a second capture mode referred to as capture mode “with structuring”, and/or
a range of current strength values and frequency values for a current to be injected into the at least one capture zone, and/or
a mode for reducing or cancelling noise contained in a captured electrical signal, which may be a first reduction mode referred to as reduction mode “without reduction”, a second reduction mode referred to as “averaging-based” reduction mode or a third reduction mode referred to as “phase opposition-based” reduction mode, and/or
a capture duration.

The imprinting parameters additionally comprise:
a selection of at least one zone of the plate used for the imprinting, referred to as imprinting zone, and/or
an imprinting mode as being a first imprinting mode referred to as imprinting mode “without structuring” or a second imprinting mode referred to as imprinting mode “with structuring”, and/or
a range of current strength values and frequency values for a current to be injected into the imprinting zone, and/or
a mode for reducing or cancelling noise contained in the signal, as being a first reduction mode referred to as reduction mode “without reduction”, or a second reduction mode referred to as “averaging-based” reduction mode or a third reduction mode referred to as “phase opposition-based” reduction mode, and/or
a type of dilutions applied to the digital signature, as being a mode referred to as mode “without dilutions”, or a mode referred to as “harmonic dilutions” mode and/or a mode referred to as “inverted harmonic dilutions” mode, and/or
a mode for applying dilutions, as being sequential or simultaneous, and/or
a volume of a neutral solution to be imprinted, for example 25 milliliters, 5 liters, 10 liters, 25 liters, and/or
an imprinting duration.

The appended drawings show, by way of example, one embodiment of a capture and imprinting device according to the invention, one mode of execution of a capture method, one mode of execution of an imprinting method and one mode of execution of a manufacturing method.

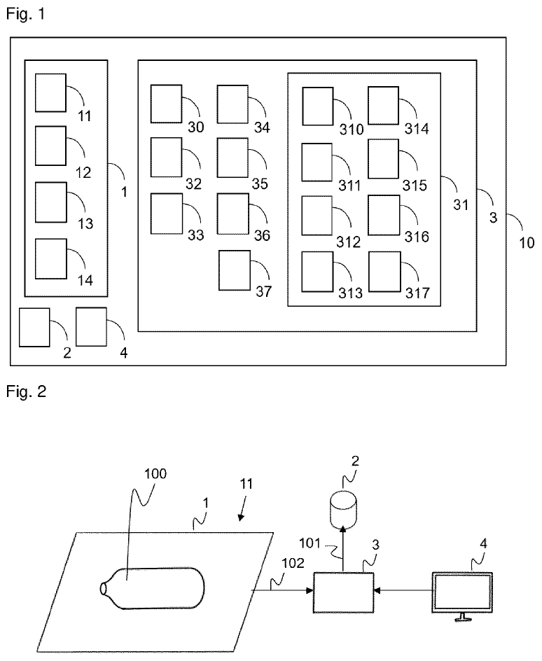

FIG. 1 schematically shows one embodiment of a capture and/or imprinting device.

FIG. 2 is a schematic description of the operation of the capture device.

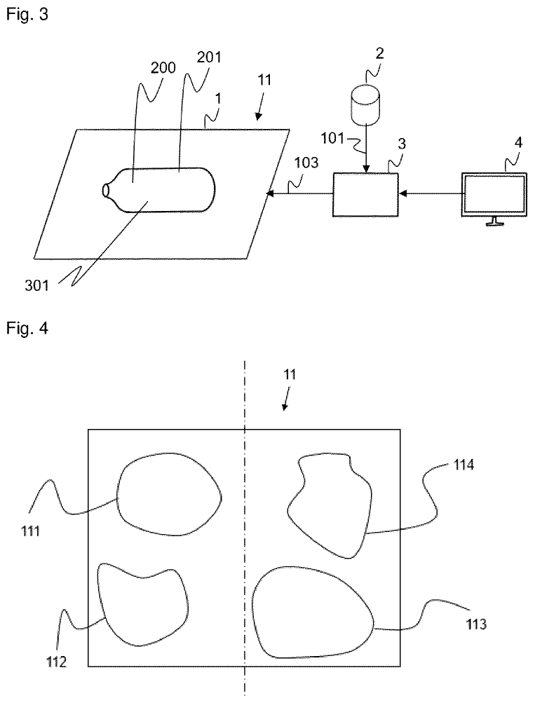

FIG. 3 is a schematic description of the operation of the imprinting device.

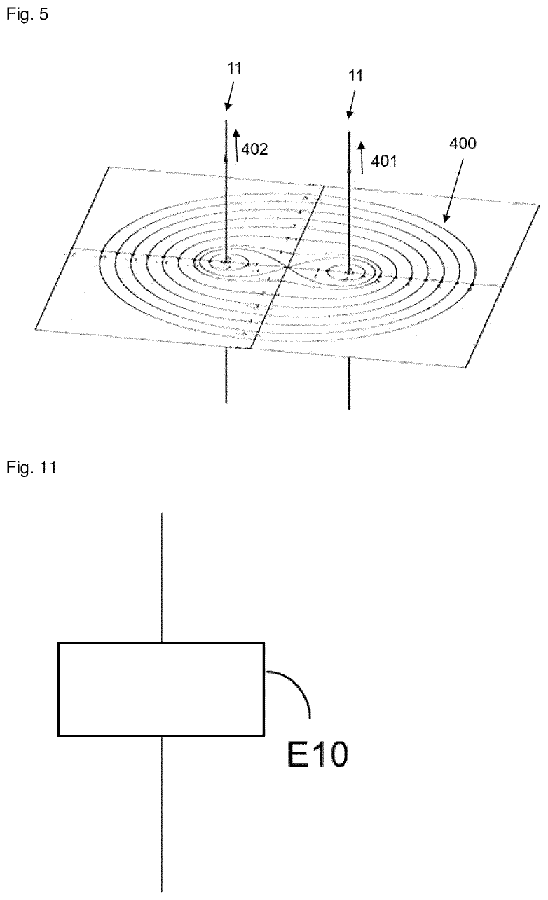

FIG. 4 is an illustration of the treatment plate according to the embodiment of the invention.

FIG. 5 illustrates one example of a virtual coil generated by two electrical wires.

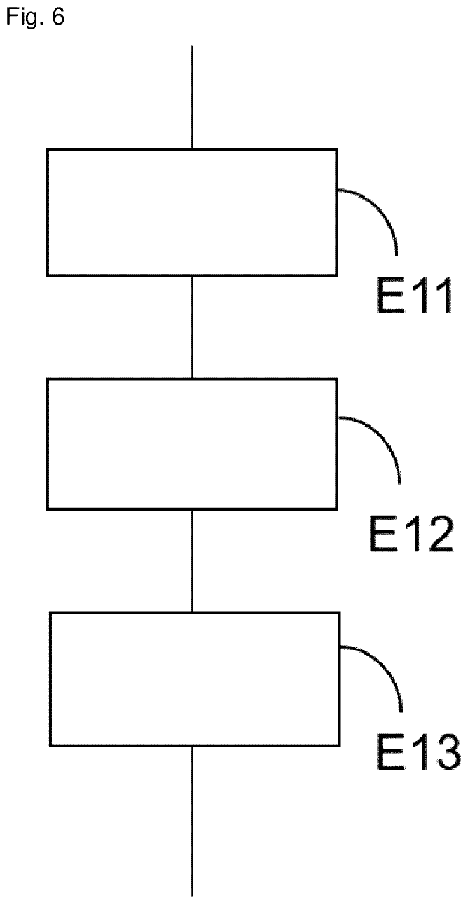

FIG. 6 is a flowchart of a capture method according to one embodiment of the invention.

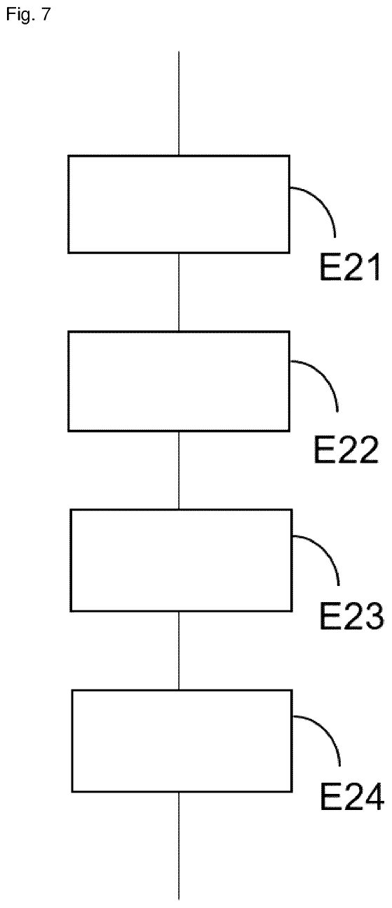

FIG. 7 is a flowchart of an imprinting method according to one embodiment of the invention.

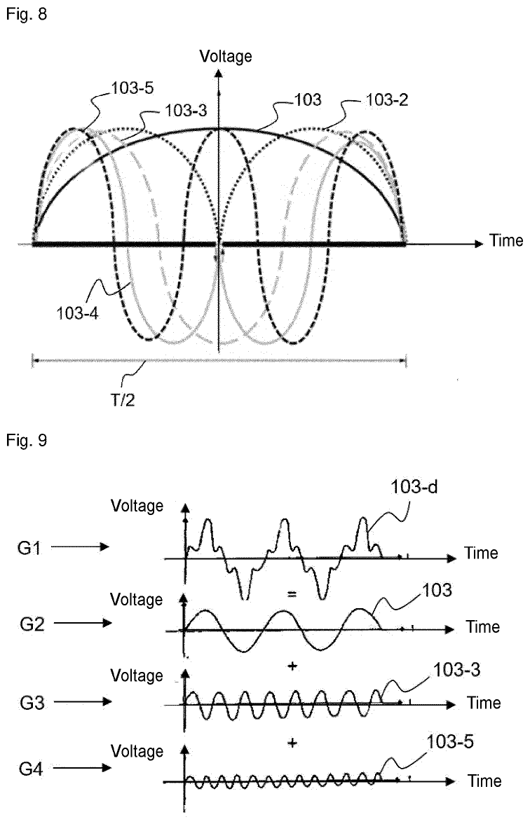

FIG. 8 illustrates harmonics of an electrical signal.

FIG. 9 illustrates simultaneous additions of harmonics to an electrical signal.

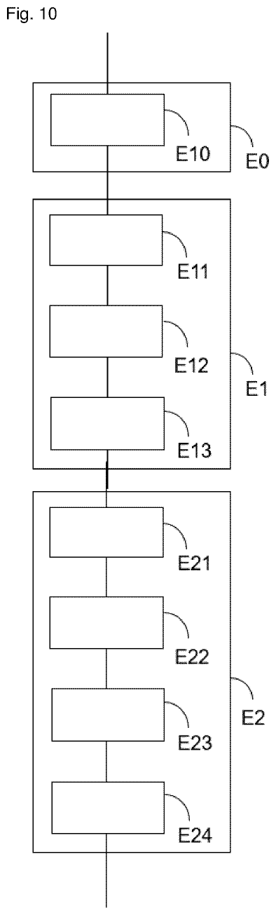

FIG. 10 is a flowchart of a manufacturing method according to the invention.

FIG. 11 is a flowchart of a structuring method according to one embodiment of the invention.

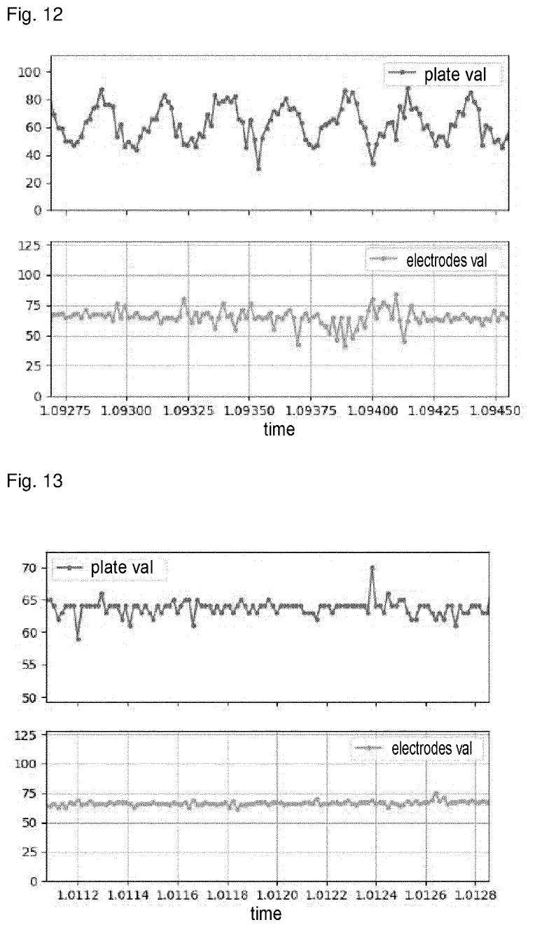

FIG. 12 shows measurements of an electrical signal as a function of time, resulting from an electromagnetic field, respectively by first electrodes and by second electrodes of a capture device according to one embodiment of the invention in the presence of a low-frequency generator.

FIG. 13 shows measurements of an electrical signal as a function of time respectively by the first electrodes and by the second electrodes of the capture device according to the embodiment of the invention in the absence of a low-frequency generator.

One exemplary embodiment of a capture and/or imprinting and/or structuring device is described below with reference to FIG. 1.

In the remainder of the document, the capture and/or imprinting and/or structuring device is called device 10.

In addition, in the remainder of the document, the information captured by the device 10 from a reference product is called “digital signature”, the term “digital” referring to the way in which the information is recorded, in a database stored in a digital memory, which is also called electronic memory in the remainder of the document.

The device 10 primarily comprises the following elements:
a treatment plate 1, also called “plate 1” in the remainder of the document,
access to a database 2;
a control unit 3;
a human-machine interface 4.

The control unit 3 incorporates at least one computer 30 and a connection system 32 allowing the computer 30 to be connected to electrodes 13, 14 of the device 10, in particular measuring electrodes 13 and injection electrodes 14 arranged on the plate 1. The control unit 3 furthermore comprises a data recording medium or electronic memory 31. It also comprises a power source 34, connected to all or some of the injection electrodes 14, thus making it possible to inject electric current into the plate 1 via the electrodes 14. It furthermore comprises at least one voltage sensor 36 for collecting voltage measurements via all or some of the measuring electrodes 13 placed on the plate 1. The electronic memory 31 makes it possible to store the measurements and/or the data computed locally in the device 10. In one embodiment, the control unit 3 may also comprise an amplifier 37 for amplifying the signals measured by the voltage sensor 36 at the terminals of all or some of the measuring electrodes 13.

The control unit 3 furthermore comprises communication means for communicating with the human-machine interface 4, and processing means for processing commands from the human-machine interface 4.

The control unit 3 comprises a link to the database 2 for storing and/or reading data in/from the database 2. In the remainder of the document, the term “request” may be used to designate a command sent by the control unit 3 to the database 2.

The database 2 contains a recording of the digital signatures of reference products 100 that have been captured.

In one embodiment, the database 2 furthermore contains a recording of the digital signatures of complex products resulting from recaptures, that is to say signatures of products that result from an imprinting phase carried out previously via the device 10.

The database 2 allows the control unit 3 to access its contents in at least two modes:
a read mode, via read requests issued by the control unit 3, and
a write mode, via write requests issued by the control unit 3.

Access to the database 2 in read mode primarily concerns an imprinting phase, which is described in the remainder of the document. Access to the database 2 in write mode primarily concerns a capture phase, which is described in the remainder of the document.

The data in the electronic memory 31 are able to be read by the computer 30, on which there is recorded a computer program for the operation of the device 10, in particular for the implementation of the capture, imprinting and manufacturing methods described in the remainder of this document.

The device 10 has the function of capturing the active effect of a reference product by way of a digital signature 101 of the reference product, in order then to be able to imprint the active effect of the reference product 100 into a treatment product 200. In addition or as an alternative, the device 10 has the function of structuring the molecules of the reference product 100 or of the treatment product 200.

FIG. 2 is a schematic description of the operation of the device 10 during a processing operation of capturing a digital signature 101 of a reference product 100.

During the capturing processing operation, the device 10 has the first function of capturing the digital signature 101 of a reference product 100 and of recording it in the database 2.

A reference product 100 may take various forms. This may in particular be a molecule, or a solution, for example an aqueous solution, a plant, a mineral or a powder.

A reference product 100 preferably has an active effect. In other words, the reference product 100 preferably comprises a bioactive compound having a therapeutic or preventive effect.

A digital signature, defined beforehand as being the information captured by the device 10 from a reference product, is said to be simple when the reference product contains a single component; as an alternative, it is said to be complex when the reference product contains multiple components.

During the capturing processing operation, a reference product 100 is placed on the plate 1 of the device 10. The control unit 3 then records an electrical signal 102 emitted by the reference product in a set 11 of electrical wires of the plate 1.

The electrical signal 102 is a superposition of vibratory signals emitted by molecules contained in the reference product, each type of molecule emitting a different vibratory signal. The vibratory signals are not necessarily sinusoidal. Water molecules contained in the reference product organize themselves into “coherence domains”, a coherence domain being a set of water molecules vibrating at the same frequency and with the same phase. The electrical signal 102 may also contain vibratory signals originating from the space between the molecules of the reference product.

In one embodiment, the electrical signal 102 is captured in the form of a voltage measured between at least two measuring electrodes 13 of the plate 1.

The voltage measured between the at least two measuring electrodes 13 of the plate 1 is transmitted to the control unit 3, which comprises processing means for processing the electrical signal 102. The control unit 3 makes it possible in particular to apply a Fourier transform analysis to the electrical signal 102. The frequency spectrum thus obtained is contained within the digital signature 101 of the reference product 100. The digital signature 101 is recorded in the database 2.

During the capture phase, in particular at the start of the capture phase, the human-machine interface 4 allows a user to parameterize the capture process, for example to parameterize the duration of the capture phase. The parameters relating to the capture and able to be configured via the human-machine interface 4 will be described later in this document.

The device 10 has the second function of imprinting an active effect into a treatment product 200, based on a digital signature 101 of a reference product 100, the digital signature 101 having been recorded beforehand in the database 2.

FIG. 3 provides a schematic description of the operation of the device 10 during a processing operation of imprinting a digital signature 101 into a treatment product 200.

The treatment product 200 is generated from an imprinting medium 201. The imprinting medium 201 is preferably a neutral medium, such as an aqueous liquid placed in a container 301. The container may be for example a receptacle 301, for example a bottle, or a patch 302.

As an alternative, the medium placed in a container may be an ionic medium, for example magnesium chloride MgCl.

The imprinting medium is placed on the plate 1. The control unit 3 retrieves a digital signature 101 from the memory 2, in order to transform it into an electrical signal 103. The electrical signal 103 is transmitted to the plate 1 by the control unit 3, in the form of a temporal variation of the voltage applied between injection electrodes 14 of the plate 1. The electrical signal commanded by the control unit 3 generates an electromagnetic field that will structure the molecules of the imprinting medium 201 and/or transfer information to them, thus generating a treatment product 200.

The structured molecules may belong to one or more coherence domains. The structuring may also apply to the space between the molecules, to particles or else to protons and/or photons emitted by the atoms of the molecules.

At the end of the imprinting processing operation, the treatment product 200 emits a digital signature 101 containing the signature of a reference product 100 used during a capture.

During the imprinting phase, in particular at the start of the imprinting phase, the human-machine interface 4 allows a user to parameterize the imprinting process, for example to parameterize the duration of the imprinting phase. The parameters relating to the imprinting and able to be configured via the human-machine interface 4 will be described later in this document.

The treatment plate 1, controlled by the control unit 3, thus makes it possible firstly to collect a digital signature 101 of a reference product 100, and secondly to inject a digital signature 101 (more precisely an active effect corresponding to this digital signature) into an imprinting medium 201 in order to obtain a treatment product 200.

The treatment plate 1, also called “plate 1” in the remainder of the document, comprises a single wire or a set 11 of electrical wires. Depending on the embodiment, the number of electrical wires in the set 11 may vary.

In one embodiment, the set 11 of wires passes through a layer 12 made of PCB material. The wires of the set 11 are electrically conductive; for example, they are copper wires.

The treatment plate 1 is intended to receive containers of various sizes, for example cylindrical bottles of 20 milliliters, 5 liters, 10 liters or 25 liters. Advantageously, the number of wires in the set 11 and their distribution must be determined so as to be consistent with the dimensions of the containers. Preferably, the distance between two contiguous wires varies in the same set of wires 11. In other words, the space between two contiguous wires of the same treatment plate 1 is variable, in order to make it possible to generate an equivalent electromagnetic field for containers of various sizes.

As illustrated in FIG. 4, the plate may comprise at least one or at least two zones 111, 112, 113, 114, such that each of the wires in the zone is contained entirely within one of the at least one or at least two zones; furthermore, in each of the at least one or at least two zones, a measured distance between each pair of contiguous wires is fixed, the distance being measured perpendicular to the direction of the wires of the pair of wires.

Furthermore, the plate may comprise a first and second zone, such that a first distance measured between each pair of contiguous wires in the first zone differs from a second distance measured between each pair of contiguous wires in the second zone.

Thus, wires in the at least one or at least two zones of the plate are capable of generating equivalent electric fields for imprinting the same active effect in various volumes of a neutral solution.

Such a differentiated distribution of the electrical wires in distinct zones of the plate 1 makes it possible to generate equivalent electromagnetic fields for containers of various sizes. In one embodiment,
for a bottle with a capacity of 20 milliliters, between 10 and 50 wires will be used,
for a bottle with a capacity of 5 liters, between 50 and 105 wires will be used,
for a bottle with a capacity of 10 liters, between 100 and 120 wires will be used, and
for a bottle with a capacity of 25 liters, between 120 and 130 wires will be used.

Adjusting the number of electrical wires used and adjusting the distance between two adjacent wires makes it possible to generate a virtual coil tailored to the size of a bottle.

FIG. 5 illustrates one example of a virtual coil 400 generated by two electrical wires through which electric currents 401, 402 respectively flow in the same direction.

When a zone 111, 112, 113, 114 of the plate 1 is used for a capture phase, a reference product 100 is placed so as to be contained within a virtual coil generated by the electrical wires in said zone. The virtual coil is then used to record the electromagnetic signals emitted by the reference product 100.

The device 10 may also be used in structuring mode. The structuring mode may be used on its own or in combination with the capture mode or the imprinting mode.

In the remainder of the document, the terms “structuring zone” or “capture zone” or “imprinting zone” are respectively used to designate at least one plate zone used for a structuring, capture or imprinting phase.

When the device 10 is used in structuring mode for structuring a medium containing water molecules, it implements a step of emitting electromagnetic waves into said medium. In one embodiment, the electromagnetic waves used to structure the medium are emitted by the electrical wires in the structuring zone of the plate. The structuring zone may be defined according to the dimensions of a container containing the medium to be structured.

An electrical structuring signal generating an electromagnetic field may pass through the electrical wires in the structuring zone. In particular, the electrical structuring signal may have a maximum current strength of between 8 and 96 μA, or even between 10 and 20 μA.

Furthermore, the structuring electromagnetic waves may be emitted over a frequency range between 120 and 30000 Hz, or even between 1000 and 5000 Hz.

Advantageously, the medium to be structured comprises at least one salt selected from magnesium chloride and sodium chloride, and the water molecules of the medium structure themselves around crystals of the at least one salt.

Advantageously, the structuring of the medium leads to the cooling of the structured medium.

In one embodiment, the human-machine interface 4 provides means allowing a user to parameterize the structuring phase. In particular, the human-machine interface advantageously makes it possible to specify parameters for implementing the structuring, in particular a range of current strength values (in microamperes) and frequency values (in Hertz) for the current injected into the set of wires 11 of the plate 1.

When the device 10 is used in capture mode, temporal voltage variations are measured between the two ends of at least one electrical wire in the capture zone. This measurement may be carried out across the terminals of multiple electrical wires in the capture zone.

The capture may be carried out in a first mode, referred to as mode “without structuring”, in which the control unit 3 does not inject any electric current into the electrical wires of the plate 1. The electric current flowing in the wires is induced only by the electromagnetic field created by the reference product placed in the virtual coil. In this capture mode, since the voltage induced across the terminals of the wires of the plate 1 is very low, the signal measured by the voltage sensor 36 of the control unit 3 may advantageously be amplified by the amplifier 37. It should be noted that the electrical circuit containing the wires is closed.

The capture may also be carried out in a second mode, referred to as mode “with structuring”, in which the control unit 3 uses the power source 34 to inject various electric currents into the electrical wires of the plate 1.

In one embodiment, the injected electrical current may have the following characteristics:
for a structuring current with a frequency of between 125 and 1999 Hertz, the current strength will be 8 microamperes,
for a structuring current with a frequency of between 2000 and 3999 Hz, the current strength will be 16 microamperes,
for a structuring current with a frequency of between 4000 and 7999 Hz, the current strength will be 32 microamperes,
for a structuring current with a frequency of between 8000 and 17999 Hz, the current strength will be chosen to be 48, 64 or 80 microamperes,
for a structuring current with a frequency of between 18000 and 30000 Hz, the current strength will be chosen to be 80 or 96 microamperes.

Structuring frequencies of between 120 and 30000 Hz, or even between 1000 and 5000 Hz, will preferably be chosen.

When the plate 1 is used to imprint a digital signature into a treatment product, the imprinting medium 201 intended to become a treatment product is placed so as to be contained within the virtual coil generated by the electrical wires in the imprinting zone. The virtual coil is then used to transmit electromagnetic signals to the imprinting medium 201, so that it becomes a treatment product 200.

The human-machine interface 4 provides means allowing a user to parameterize the capture phase.

In one embodiment, a first parameter of the capture phase relates to the definition of the capture zone, that is to say the selection of the at least one zone of the plate 111, 112, 113, 114 that will be used for the capture.

In addition or as an alternative, a second parameter of the capture phase relates to the capture mode, which may be the first mode, referred to as mode “without structuring”, or the second mode, referred to as mode “with structuring”. When the second capture mode is selected, the human-machine interface advantageously makes it possible to specify parameters for implementing the structuring, in particular a range of current strength values (in microamperes) and frequency values (in Hertz) for the current injected into the set of wires 11 of the plate 1.

In addition or as an alternative, a third parameter of the capture phase relates to a mode for reducing or cancelling noise contained in the voltage signal measured by the control unit 3 via the tensiometer and the measuring electrodes 13. In one embodiment, the human-machine interface 4 makes it possible to
deactivate the noise cancellation processing, or
select averaging-based noise cancellation processing, or
select phase opposition-based noise cancellation processing.

Filtering an electrical signal by averaging consists in calculating the average value of the signal. A sliding average may also be calculated. In this case, the signal is averaged over a fixed number of values. For example, an average is calculated over a group of ten consecutive values, followed by an average over the group of the next ten values, and the operation is repeated over the entire signal.

In addition, the human-machine interface provides means for managing recordings of digital signatures, in particular means for
defining a duration of a recording,
starting and stopping a recording,
manually or automatically ending a recording,
organizing the storage of a recorded digital signature in the database 2.

Furthermore, the human-machine interface 4 provides means allowing a user to parameterize the imprinting phase.

In one embodiment, a first parameter of the imprinting phase relates to the definition of the imprinting zone, that is to say the selection of the at least one zone of the plate 111, 112, 113, 114 that will be used for the imprinting.

A second parameter of the imprinting phase relates to the selection, from the database 2, of at least one digital signature to be imprinted into an imprinting medium in order to obtain a treatment product 200.

In addition or as an alternative, a third parameter of the imprinting phase relates to the choice of an imprinting mode, which may be the first mode, referred to as mode “without structuring”, or the second mode, referred to as mode “with structuring”. When the second imprinting mode is selected, the human-machine interface advantageously makes it possible to specify parameters for implementing the structuring, in particular a range of current strength values and frequency values for the current injected into the set of wires 11 of the plate 1.

In addition or as an alternative, a fourth parameter of the imprinting phase relates to the choice of a mode for reducing or cancelling noise contained in the digital signature to be imprinted. In one embodiment, the human-machine interface 4 makes it possible to
deactivate the noise cancellation processing, or
select averaging-based noise cancellation processing, or
select phase opposition-based noise cancellation processing.

In addition or as an alternative, a fifth parameter of the imprinting phase relates to the selection of a volume of imprinting medium 201, that is to say the volume of treatment liquid that it is desired to obtain, for example 25 milliliters, 5 liters, 10 liters, 25 liters, etc.

In addition or as an alternative, a sixth parameter of the imprinting phase relates to a mode for constructing the electrical signal 103 that will be injected into the plate 1, in particular into the imprinting zone. In particular, the sixth parameter of the imprinting makes it possible to calibrate the processing operations that will be applied to at least one selected digital signature 101 to generate the electrical signal 103. For example, the sixth parameter may relate to the imprinting of dilutions of the digital signature. For example, depending on this parameter, the dilutions may be harmonics and/or inverted harmonics of the digital signature. Furthermore, the sixth parameter may allow a user to determine whether the harmonics may be superimposed, that is to say integrated simultaneously into the electrical signal, or else whether the harmonics should be chained, that is to say integrated successively into the electrical signal.

Advantageously, the human-machine interface 4 also allows a user to parameterize the imprinting duration.

The human-machine interface 4 also makes it possible to start and stop the imprinting phase.

In the embodiment of the invention, the computer 31 of the control unit 3 makes it possible to run software comprising the following modules, which communicate with one another:
a structuring module 310, which collaborates with the plate 1,
a recording module 311 for recording an electrical signal 102, which communicates with the plate 1 and the human-machine interface 4,
a frequency processing module 312 for carrying out frequency processing on the recorded electrical signal 102,
a storage module 313 for storing the digital signature 101, which communicates with the database 2,
a reading module 314 for reading the digital signature 101 of a reference product from the database 2, which collaborates with the database 2,
a determination module 315 for determining imprinting parameters, which collaborates with the human-machine interface 4,
a construction module 316 for constructing an electrical imprinting signal 103,
an imprinting module 317 for imprinting the active effect into a neutral solution 201, which collaborates with the plate 1.

FIG. 11 shows one mode of execution of a structuring method. The structuring method comprises a step E10. The progress of step E10 is described in the remainder of the document, in particular as a sub-step of the capture method.

One mode of execution of a method for capturing a digital signature is described below with reference to FIG. 6.

In a step E10 that may take place before and/or during and/or after steps E11 and/or E24, the water molecules contained in a product are structured, the product possibly being a reference product or a treatment product.

Step E10 comprises a sub-step E101 of parameterizing the structuring phase, in particular
parameterizing a structuring zone,
and parameterizing a current strength and a frequency of an electrical signal circulating in the structuring zone.

The sub-steps for implementing the structuring are described in the remainder of the document. In particular, sub-step E112 comprises implementing structuring, which is carried out during a capture.

In a first step E11, an electrical signal 102 measured between at least two measuring electrodes 13 of the device 10 is recorded.

The first step E11 comprises a sub-step E111 of parameterizing the recording of the signal, that is to say of parameterizing the capture phase.

The parameters of the capture phase comprise:
a capture zone, that is to say the at least one zone of the plate 111, 112, 113, 114 used for the capture,
a capture mode, with or without structuring,
where applicable, a range of current strength values and frequency values for a current to be injected into the capture zone,
a mode for reducing or cancelling noise,
a recording duration.

In one embodiment, default values for each parameter of the capture phase are stored in the local memory 31 and may be assigned to the parameters when sub-step E111 starts to be carried out. For example, the default values may correspond to the following parameterization:
all zones of the plate 1 are used for the capture,
the default capture mode is a mode without structuring,
no current is injected into the plate 1 by the control unit 3,
no noise cancellation processing is applied,
the recording duration is indefinite; the recording will be interrupted at the request of the user via the human-machine interface 4.

During sub-step E111, the parameterization requests defined by a user are processed. The parameterization requests coming from the human-machine interface 4 may relate to each of the parameters described above for the capture phase.

Upon receipt of a parameterization request, the parameters of the capture phase are updated in the local memory 31 according to the parameterization defined in the request.

The parameterization requests are thus processed again until a request to start the recording is received from the human-machine interface 4.

Advantageously, the request to start the recording starts when a reference product 100 has been placed on the plate 1.

Upon receipt of a request to start the recording, a transition then takes place to a sub-step E112 of recording an electrical signal between at least two measuring electrodes 13. The electrical signal consists of a voltage variation between the at least two measuring electrodes 13.

The parameters defined previously for the capture during sub-step E111 are then applied.

If a recording duration has been defined by the user, a time count T_RECORD with a duration equal to the recording duration defined by the user is started.

The at least two measuring electrodes 13 used for the recording are selected as a function of the zones of the plate 1 chosen by the user for this recording, this information being accessible in the local memory 31.

If the capture mode is parameterized with structuring, the recording step E11, and more particularly sub-step E112, comprises a step of structuring the reference product 200 by sending an electrical structuring signal in electrical wires of the plate 1 on which said reference product 200 is placed.

In this case, injection electrodes 14 are also selected so as to inject a current into the zones of the plate 1 chosen by the user. Furthermore, in sub-step E112, the current strength and the frequency of the structuring current are calibrated as a function of the parameters recorded in the local memory 31, these parameters possibly having been updated during sub-step E111.

The power source 34 is controlled so as to generate the structuring current defined by the user, and to transmit the structuring current to the chosen plate zones, via the selection of injection electrodes 14.

In one embodiment, the electrical signal or structuring current has a frequency of between 120 and 30000 Hz, or even between 1000 and 5000 Hz. In one embodiment, the electrical signal or structuring current has a maximum current strength of between 8 and 96 μA, or even between 10 and 20 μA.

At the same time as structuring current is injected into the plate, voltage variations are measured by the voltage sensor 36 between the terminals of the selection of measuring electrodes 13. The measured voltage variations are recorded in the local memory 31.

In one embodiment, when the recording is carried out without structuring, the voltage variations measured by the sensor 36 are amplified by the amplifier 37 before being recorded in the local memory 31.

Depending on the parameterization of the capture phase, noise reduction or cancellation processing might have been selected by the user. The noise reduction method chosen by the user, for example an averaging-based or phase opposition-based noise reduction method, may then be applied to the voltage variations measured between the at least two measuring electrodes 13. The voltage measurements will then be recorded in the local memory 31 after the noise reduction or cancellation processing.

The recording step E11 ends when one of the following conditions is met:
the user commands stopping of the recording via the human-machine interface 4, which transmits the stop instruction to the control unit 3, or
the time count T_RECORD runs out.

This is then followed by step E12 of carrying out frequency processing on the recorded electrical signal to form a digital signature.

In one embodiment, the digital signature 101 of the reference product 100 is obtained by carrying out a Fourier transform on the electrical signal 102 recorded in step E11.

The digital signature may be generated from the same initial electromagnetic recording using multiple different processing algorithms, in particular by way of:
a fast Fourier transform or FFT, and/or
a temporal signal (for example temporal variation of a voltage or current)

This is then followed by step E13 of storing the digital signature 101 in the database 2. For this purpose, in step E13, a write request is transmitted to the database 2. The request contains the digital signature 101 of the reference product 100.

One mode of execution of a method for imprinting an active effect is described below with reference to FIG. 7.

In a first step E21, the digital signature of at least one reference product 100 is extracted from the database 2.

For this purpose, a request from a user via the human-machine interface 4 is processed. The request contains a selection of at least one identifier of a digital signature 101 to be imprinted.

The at least one identifier is transmitted to the database 2 in the form of a read request.

A response is received from the database 2. The response contains the digital signatures respectively associated with each of the at least one identifier of a digital signature.

In a second step E22, the imprinting parameters are determined.

The parameters of the imprinting phase comprise:
an imprinting zone, that is to say the at least one zone of the plate 111, 112, 113, 114 used for the imprinting,
an imprinting mode, with or without structuring,
where applicable, a range of current strength values and frequency values for a current to be injected into the imprinting zone,
a mode for reducing or cancelling noise (no noise reduction, averaging-based noise reduction or phase opposition-based noise reduction),
a type of dilutions applied to the digital signature (no dilution, harmonic dilutions or inverted harmonic dilutions), and the mode for applying these dilutions (sequential or simultaneous)
a volume of imprinting medium, that is to say the volume of treatment liquid that it is desired to obtain, for example 25 milliliters, 5 liters, 10 liters, 25 liters,
an imprinting duration.

In one embodiment, default values for each parameter of the imprinting phase are stored in the local memory 31 and may be assigned to the parameters when step E22 starts to be carried out. For example, in one mode of execution, the default values correspond to the following parameterization:
all zones of the plate 1 are used for the imprinting,
the default imprinting mode is a mode without structuring,
no current is injected into the plate 1 by the control unit 3,
no noise cancellation processing is applied,
the volume of imprinting medium is 25 liters,
no dilution is applied,
the imprinting duration is indefinite; the imprinting will be interrupted at the request of the user via the human-machine interface 4.

During step E22, the parameterization requests defined by a user via the human-machine interface 4 are processed. The parameterization requests coming from the human-machine interface 4 may relate to each of the parameters described above for the imprinting phase.

Upon receipt of a parameterization request, the parameters of the imprinting phase are updated in the local memory 31 according to the parameterization defined in the request.

The parameterization requests are thus processed again until a request to start the imprinting is received from the human-machine interface 4.

Advantageously, the request to start the recording is received after an imprinting medium has been placed on the plate 1.

Upon receipt of a request to start the imprinting, a transition then takes place to step E23, in which an electrical imprinting signal is constructed from the at least one digital signature 101.

In one embodiment, the at least one digital signature 101 recorded in memory is a temporal signal on the basis of which the electrical signal 103 is generated.

The electrical signal 103 that is obtained is then processed in accordance with the parameters specified by the user in step E22.

Depending on the parameterization of the imprinting phase, noise reduction or cancellation processing might have been configured by the user. The noise reduction method chosen by the user, for example an averaging-based or phase opposition-based noise reduction method, may then be applied to the electrical signal 103.

Furthermore, if the user has selected a type of dilutions to be applied to the digital signature, then the electrical signal 103 may be modified according to various options. The addition of harmonics has the effect of improving the efficiency of the processing.

If harmonic dilutions are involved, harmonics of the electrical signal 103 will be added to the original electrical signal 103, according to the parameterization chosen by the user.

FIG. 8 shows harmonics of an electrical signal 103 over a half-period T/2:
the curve 103-2 represents the second harmonic: its frequency is twice that of the signal 103,
the curve 103-3 represents the third harmonic: its frequency is three times that of the signal 103,
the curve 103-4 represents the fourth harmonic: its frequency is four times that of the signal 103,
the curve 103-5 represents the fifth harmonic: its frequency is five times that of the signal 103.

The harmonics may be added simultaneously; in this case, the harmonics are superimposed on the original electrical signal 103. As an alternative, the harmonics may be added sequentially; in this case, the harmonics are integrated following the original electrical signal 103.

The principle of simultaneously adding harmonics to an electrical signal 103 is illustrated by the graphs G1 to G4 in FIG. 9:
the graph G2 illustrates an electrical signal 103, which is a sinusoidal signal with a period of 20 milliseconds; the signal 103 has a fundamental frequency of 50 Hertz,
the graph G3 represents the signal 103-3, which is the third harmonic of the signal 103, that is to say the frequency of which is three times the fundamental frequency,
the graph G4 represents the signal 103-5, which is the fifth harmonic of the signal 103, that is to say the frequency of which is five times the fundamental frequency,
the graph G1 represents the signal 103-d, corresponding to a signal 103 integrating the third and fifth harmonics simultaneously, that is to say to the signal 103 plus its third and fifth harmonics simultaneously.

In addition or as an alternative, the user may choose inverted harmonic dilutions. The inverted harmonics correspond to frequency dividers dividing the frequency of the electrical signal 3. The inverted harmonics may also be added sequentially or simultaneously.

At the end of step E23, an electrical imprinting signal 103 has been determined. Depending on the options chosen by the user, the electrical imprinting signal may be a filtered signal (to eliminate noise therefrom); in addition or as an alternative, the electrical imprinting signal may or may not integrate dilutions, sequentially or simultaneously.

In a fourth step E24, the active effect of the at least one reference product is imprinted into a neutral solution comprising at least one salt, by passing the electrical imprinting signal 103 through a set of electrical wires of a plate of an imprinting device on which the neutral solution is positioned, for the predetermined imprinting duration, so as to imprint the active effect of the at least one reference product into the neutral solution and thus transform it into a treatment product.

In one embodiment, the at least one salt is selected from sodium chloride and/or magnesium chloride.

Furthermore, the neutral solution may be or may comprise sand, sugar beads, glass or else various polymers.

The neutral solution comprising at least one salt is an imprinting medium into which the active effect of the reference product is imprinted.

At the start of step E24, the imprinting medium must be placed on the plate 1. If the user has selected an imprinting zone via the human-machine interface 4, then the imprinting medium must be positioned on the imprinting zone selected by the user.

In one embodiment, the imprinting zone may be determined as a function of the volume of the imprinting medium 201 to be imprinted and/or the size of the container 301.

If an imprinting duration has been defined by the user, in step E24, a time count T_IMP with a duration equal to the imprinting duration defined by the user is started.

The power source 34 is then controlled so as to generate the electrical imprinting signal 103 and to transmit the electrical imprinting signal 103 to the imprinting zone, via a selection of injection electrodes 14.

If the imprinting mode is parameterized with structuring, the imprinting step E24 comprises a step of structuring the imprinting medium by sending an electrical structuring signal in electrical wires of the plate 1 on which the imprinting medium is placed, or in the electrical wires in the imprinting zone on which the imprinting medium is placed.

In this case, injection electrodes 14 are also selected so as to inject a structuring current onto all or part of the plate 1. In addition, the current strength and the frequency of the structuring current are calibrated as a function of the parameters recorded in the local memory 31.

The power source 34 is controlled so as to generate the structuring current defined by the user, and to transmit the structuring current to the chosen plate zones, via a selection of injection electrodes 14.

In one embodiment, the electrical signal or structuring current has a frequency of between 120 and 30000 Hz, or even between 1000 and 5000 Hz. In one embodiment, the electrical signal or structuring current has a maximum current strength of between 8 and 96 μA, or even between 10 and 20 μA.

The imprinting step E24 ends when one of the following conditions is met:
the user commands stopping of the imprinting via the human-machine interface 4, which transmits the stop instruction to the control unit 3, or
the time count T_IMP runs out.

One mode of execution of a method for manufacturing a treatment product is described below with reference to FIG. 10.

In a first step E1, at least one digital signature of a reference product is captured. Step E1 comprises sub-steps E11, E12 and E13 described above, which are carried out successively.

Next, in a second step E2, at least one active effect is imprinted into a neutral solution. Step E2 comprises sub-steps E21, E22, E23 and E24 described above, which are carried out successively.

The method for manufacturing a treatment product may comprise an additional phase E0 in which a medium containing atoms or molecules having the ability to organize themselves, for example a medium containing water molecules, is structured. The additional phase E0 comprises sub-step E10 described above. In addition, the additional phase E0 may be carried out before and/or during the first capture phase E1, in order to structure the reference solution, and/or the additional phase E0 may be carried out before and/or during the second imprinting phase E2, in order to structure the treatment product.

Experiments were carried out to empirically illustrate some aspects of the concept of the invention.

According to a first experiment, the existence of a difference between a structured and an unstructured solution is highlighted. In this first experiment, twenty vials each containing 100 ml of sterilized deionized water mixed with magnesium chloride MgCl2 were prepared. Measurements were taken five times for each vial, before and after the implementation of a structuring phase as described above. A photoplethysmograph, applied directly through the vial, was used for these measurements. This device consists of a PPG optical sensor equipped with three LEDs (one green 536 nm, one red 660 nm and one infrared 940 nm) and two photodiodes (sampling rate 100 Hz, pulse width 115.2 ms).

Absorbance rates were recorded for these three different wavelengths: 536 nm (green), 660 nm (red) and 940 nm (infrared).

To guarantee repeatability of the measurements, we calculated the coefficient of variation for each raw signal, as defined by equation 1 below:

$$Coefficient of variation ( CV ) \% = ( Standard deviation / average ) \times 100 \qquad ( Equation 1 )$$

In addition, to analyze the magnitude of the changes in absorbance before and after structuring, we first examined whether the data followed a normal distribution. Next, we applied Fisher's parametric test, deriving p-values to determine levels of significance: insignificant (p>0.05), first degree of significance (*, p<0.05), second degree (**, p<0.01), and third degree (***, p<0.001).

An algorithm was developed to estimate water state, based on a binomial distribution presented in equation 2 below:

$$Water state ( structured level ) = 1 / 1 + {e}^{⋀} ( - ( a + bx 1 + cx 2 + … + ixn ) ) ) \qquad ( Equation 2 )$$

where a, b, . . . , i are constants and x1, x2, . . . , xn represent experimental variables.

With fewer variables in this study, we used a simplified logistic binomial distribution. Its density function is given by equation 3 below:

$$f ( x ; μ , s ) = [ 1 / {s ( 1 + {e}^{⋀} ( - ( x - μ ) / s ) )}^{⋀} 2 ] \qquad ( Equation 3 )$$

μ and s being parameters of the experiment and x being the variable (measured absorbance).

The corresponding distribution function is expressed by equation 4 below:

$$F ( x ; μ , s ) = 1 / ( 1 + {e}^{⋀} ( - ( ( x - μ ) / s ) ) ) \qquad ( Equation 4 )$$

In this study, it was observed that the expected expectation value E(X)=μ=0 and the variance Var(X)=(s²π²)/3, leading to s=1. The final distribution function of the score for the water state is then represented by equation 5:

$$F ( x ) = 1 / ( 1 + {e}^{⋀} ( - x ) ) \qquad ( Equation 5 )$$

where x corresponds to the measured absorbance.

The algorithm was initially created on the basis of 25 observations and was then validated on an independent subgroup of 15 observations.

### Repeatability of the Measurements:

For unstructured water, variability was 0.076% at 536 nm, 0.055% at 660 nm and 0.068% at 940 nm.
For structured water, variability was 0.070% at 536 nm, 0.041% at 660 nm and 0.528% at 940 nm.

### Differences in Water Absorption Before and After Structuring:

Applying Fisher's tests to these data, respectively before and after structuring, gave the results (NS meaning “not significant”) presented in Table 1 below.

| Variable | Fisher p-value |
|---|---|
| Green 536 nm | 0.001, *** |
| Red 660 nm | 0.966, NS |
| Infrared 940 nm | 0.230, NS |

A significant difference in absorbance (p=0.001, ***) was observed only at 536 nm, indicating a change due to dynamization. The calculated Fisher p-value is lower than the significance threshold of p=0.05, thereby allowing the assumption that the ratio of variances is other than 1.

### Wavelength Variation Ratio:

Like phenomena observed in human blood, some changes are not directly visible in the raw wavelength data. By calculating the ratio of red/infrared and red/green absorbance variations, significant changes were quantified. The obtained values are summarized in the table below.

| | Ratio value for | Ratio value for | Fisher |
|---|---|---|---|
| Variable | structured water | unstructured water | p-value |
| Red/Infrared | 0.642 ± 0.144 | 0.619 ± 0.040 | <0.0001, *** |
| Red/Green | 1.576 ± 0.073 | 1.534 ± 0.082 | <0.0001, *** |
| Infrared/Green | 2.507 ± 0.258 | 2.484 ± 0.165 | 0.015, * |

The red/infrared and red/green ratios in structured water are significantly higher than in the unstructured samples. Even the infrared/green ratio showed a significant increase in structured water.

### Water State Prediction:

These observations make it possible to predict the level of structuring of the water tested, as shown in the tables below.

| | | | Prediction |
|---|---|---|---|
| | | | of the water |
| | | | status |
| Obser- | | | (structuring |
| vations | | Prediction | level) % |
| 2 | Unstructured | Unstructured | 4.9 |
| 6 | Unstructured | structured | 99.8 |
| 9 | Unstructured | Unstructured | 46.0 |
| 10 | Unstructured | structured | 100.0 |
| 13 | Unstructured | structured | 90.9 |
| 15 | Unstructured | Unstructured | 23.4 |
| 16 | Unstructured | Unstructured | 29.1 |
| 18 | Unstructured | structured | 100.0 |
| 19 | Unstructured | structured | 100.0 |
| 23 | structured | structured | 100.0 |
| 30 | structured | structured | 98.8 |
| 31 | structured | structured | 82.1 |
| 33 | structured | structured | 99.2 |
| 37 | structured | structured | 85.4 |
| 39 | structured | structured | 56.6 |

| | | | | % |
|---|---|---|---|---|
| From/to | structured | Unstructured | Total | Correct |
| structured | 6 | 0 | 6 | 100.00% |
| Unstructured | 5 | 4 | 9 | 44.44% |
| Total | 11 | 4 | 15 | 66.67% |

As indicated, the prediction was 100% accurate for structured water. However, there were five false positives in unstructured water samples, leading to an overall prediction accuracy of 66.7%. This performance is based on absorbance measurements at 536 nm, 660 nm and 940 nm. The five false positives out of nine in the prediction for structured water in unstructured samples suggest the need for a larger sample to improve the robustness of the algorithm. In addition, the presence of structured water on the surface of the glass vials might have influenced some wavelength readings, leading to inaccurate predictions.

We observed acceptable variability (less than 0.1%) over all wavelengths before and after structuring, except at 940 nm after structuring. This anomaly is attributed to photon emission resulting from water restructuring, which potentially affects this wavelength.

Given the absorption properties of oxyhemoglobin and deoxyhemoglobin, it appears that free water and structured water absorb different wavelengths differently. This variance may be related to changes in hydrogen bonds, as indicated by the WAMACS method at higher wavelengths.

An algorithm developed to predict the presence of structured water achieved an average accuracy of 66.7%, although it also produced five false positives in unstructured water samples. In any case, this initial work is sufficient to demonstrate the existence of a difference between structured and unstructured water, and the possibility of identifying water state. The points discussed above show that the accuracy may tend toward the 100% prediction. The experiment highlights the difference between structured and unstructured vials, which do not react in the same way to the given wavelengths. The teaching of this experiment is to highlight this phenomenon. It is not necessary to detect the presence of structured or unstructured water for a given solution to implement the invention in practice.

A second experiment is carried out to highlight the possibility of carrying out capturing using a capture device according to one embodiment of the invention. For this purpose, a low-voltage generator is positioned on the plate of such a capture device according to one embodiment of the invention, and a 4 kHz (sinusoidal) signal is generated.

FIG. 12 shows measurements of an electrical signal, resulting from an electromagnetic field, as a function of time respectively by first electrodes and by second electrodes of the capture device according to the embodiment of the invention in the presence of the low-frequency generator. The first electrodes, referred to as plate electrodes, which correspond to the measuring electrodes 13 described above, measure the electrical values referred to as “plate val”. These values form the first curve of FIG. 12, on which there is indeed obtained a signal with a frequency of 4 kHz that corresponds to the signal transmitted to the capture device by the low-voltage generator. This result demonstrates that the capture device is capable of capturing such an electromagnetic electrical signal. It should be noted that this electrical signal is slightly distorted by the inevitable presence of noise. To better identify the noise, additional electrodes of the capture device, which are positioned further away from the plate, also measure a received electrical signal (electrodes val), which is reproduced by the second curve of FIG. 12. It appears that these electrodes do not (or barely) receive the signal from the low-voltage generator, and that the obtained signal ultimately corresponds to the surrounding noise. On this basis, it is possible to reprocess the signal measured by the first electrodes of the plate, by removing from it the noise as captured by the additional electrodes.

FIG. 13 shows the same measurements of an electrical signal as a function of time as in the case of FIG. 12, but in the absence of any signal emitted by a low-voltage generator. The electrodes of the plate then measure a signal very different from that of FIG. 12, thereby confirming that they are capable of specifically capturing a signal emitted at the plate, where they are positioned.

Finally, a third experiment is carried out to validate the method for manufacturing a treatment product, including the capture method and the imprinting method. For this purpose, eight specific products were manufactured by imprinting a signal, according to the described manufacturing method. For each product, 20 captures were carried out by the capture device according to one embodiment of the invention, and recorded in a database in the form of reference signals. As a variant, any other number of captures could be carried out for each product, one capture, two captures or any number greater than two captures. Carrying out multiple captures makes it possible to obtain a larger initial source of information and to facilitate future processing operations. In the experiment carried out, the database thus comprises 160 recordings. Next, a large number of measurements is carried out for various products by the capture device according to the embodiment of the invention, these measurements involving, in 19 situations distributed randomly among the large number of measurements, a certain product out of the eight that were initially chosen. For each measurement from this large number of measurements, the capture is compared with the reference signals. This comparison made it possible to accurately identify the 19 measurements of said certain product out of the eight. In other words, this experiment made it possible to demonstrate that a certain product does indeed comprise a reliably identifiable digital signature, thereby validating both the principle of capturing a signal and the principle of imprinting a signal.

1. A method for imprinting an active effect of at least one reference product into a treatment product, wherein the method comprises a second imprinting phase, the second imprinting phase comprising:
reading an associated digital signature of the at least one reference product from a database;
constructing an electrical imprinting signal based on the digital signature of the reference product; and
imprinting an active effect into a neutral solution comprising at least one salt, by passing the electrical imprinting signal through one or more electrical wires of a plate of an imprinting device on which the neutral solution is positioned, for a predetermined imprinting duration, so as to imprint the active effect of the at least one reference product into the neutral solution and thus transform the active effect of the at least one reference product into a treatment product.

2. The method as claimed in claim 1, wherein the plate comprises
at least two substantially rectilinear and parallel wires, or
at least one wire in the shape of a spiral.

3. The method as claimed in claim 1, wherein the method further comprises:
determining imprinting parameters, comprising a chosen imprinting duration, a duration for which the imprinting of the active effect is carried out being equal to the chosen imprinting duration.

4. The method as claimed in claim 1, wherein the constructing of the electrical imprinting signal based on the digital signature of the reference product comprises:
obtaining a first electrical signal by applying an inverse Fourier transform to the at least one digital signature of the reference product.

5. The method as claimed in claim 1, wherein the method further comprises:
structuring the neutral solution,
the structuring being carried out before and/or during the second imprinting, by implementing a structuring method comprising emitting electromagnetic waves into the neutral solution.

6. The method as claimed in claim 5, wherein the neutral solution comprises at least one salt selected from the group consisting of magnesium chloride and sodium chloride, wherein water molecules structure themselves around crystals of the at least one salt.

7. The method as claimed in claim 5, wherein the electromagnetic waves of the structuring method are emitted over a frequency range of from 120 to 30,000 Hz.

8. The method as claimed in claim 5, wherein the electromagnetic waves of the structuring method are generated by an electrical structuring signal having a maximum current strength in a range of from 8 to 96 μA.

9. A method of manufacturing a treatment product, wherein the method comprises:
firstly, capturing at least one digital signature of a reference product by implementing a capture method comprising:
recording an electrical signal measured between at least two measuring electrodes of a capture device comprising a plate on which the reference product is positioned, the plate comprising one or more electrical wires capable of generating a virtual coil on a volume containing the reference product;
processing the recorded electrical signal to form a digital signature comprising a frequency spectrum of the recorded electrical signal; and
storing the digital signature in a database, and
secondly, imprinting at least one active effect into a neutral solution based on the digital signature by implementing the imprinting method as claimed in claim 1.

10. A device for imprinting an active effect, wherein the device comprises:
a treatment plate comprising a set of one or more electrical wires;
an access to a database comprising a storage of a digital signature of at least one reference product;
a control unit comprising hardware and software configured to implement an imprinting method comprising a second imprinting phase(E2), the second imprinting phase comprising the following steps:
reading an associated digital signature of the at least one reference product from a database;
constructing an electrical imprinting signal based on the digital signature of the reference product; and
imprinting an active effect into a neutral solution comprising at least one salt, by passing the electrical imprinting signal through one or more electrical wires of a plate of an imprinting device on which the neutral solution is positioned, for a predetermined imprinting duration, so as to imprint the active effect of the at least one reference product into the neutral solution and thus transform it the active effect of the at least one reference product into a treatment product,
the control unit comprising a link, firstly to the database, the link being adapted for storing and/or reading the at least one digital signature in/from the databases, and secondly to the set of one or more electrical wires of the treatment plate, the link being adapted for generating and/or measuring an electrical signal; and
a human-machine interface adapted for starting and/or interrupting the reading and/or the capturing and/or the imprinting, and for inputting structuring and/or capturing and/or imprinting parameters.

11. The device as claimed in claim 10, wherein the device further comprises:
at least a first measuring and/or injection electrode and a second measuring and/or injection electrode,
the one or more electrical wires of the set of one or more electrical wires of the plate being connected to the first measuring and/or injection electrode and the second measuring and/or injection electrode in an electrical circuit,
a first end of each of the one or more electrical wires being connected to the first measuring and/or injection electrode and a second end of each of the one or more electrical wires being connected to the second measuring and/or injection electrode.

12. The device as claimed in claim 10, wherein the one or more electrical wires of the set of one or more electrical wires of the treatment plate are
parallel to and equidistant from one another, or
in the shape of a spiral.

13. The device as claimed in claim 12, wherein the treatment plate comprises:
a plurality of the electrical wires, and
a first zone and second zone,
a first distance measured between each pair of contiguous electrical wires in the first zone differs from a second distance measured between each pair of contiguous electrical wires in the second zone.

14. The device as claimed in claim 12, wherein the electrical wires in at least one zone of the treatment plate are capable of generating equivalent electric fields for imprinting the active effect in various volumes of a neutral solution.

15. The device as claimed in claim 10, wherein the imprinting parameters comprise:
a selection of at least one zone of the plate used for imprinting, referred to as imprinting zone, and/or
a first imprinting mode referred to as imprinting mode without structuring or a second imprinting mode referred to as imprinting mode with structuring, and/or
a range of current strength values and frequency values for a current to be injected into the imprinting zone, and/or
a first reduction mode for reducing or cancelling noise contained in the electrical imprinting signal “without reduction” electrical imprinting signal without reduction, or a second reduction mode, which is an averaging-based reduction mode for reducing or cancelling noise contained in the electrical imprinting signal, or a third reduction mode which is a phase opposition-based reduction mode for reducing or cancelling noise contained in the electrical imprinting signal, and/or
a mode without dilutions applied to the digital signature, a harmonic dilutions mode, and/or an inverted harmonic dilutions mode, and/or
a mode for applying sequential or simultaneous dilutions, and/or
a volume of a neutral solution to be imprinted, and/or
an imprinting duration.

16. The method as claimed in claim 4, wherein the constructing of the electrical imprinting signal based on the digital signature of the reference product comprises:
generating a set of harmonics of the first electrical signal, and then adding the set of harmonics to the first electrical signal, the set of harmonics comprising second and/or third and/or fourth and/or fifth harmonics.

17. The method as claimed in claim 7, wherein the electromagnetic waves of the structuring method are emitted over a frequency range of from 1,000 to 5,000 Hz.

18. The method as claimed in claim 8, wherein the electromagnetic waves of the structuring method are generated by an electrical structuring signal having a maximum current strength in a range of from 10 to 20 μA.

19. The method as claimed in claim 2, wherein the method further comprises:
determining imprinting parameters, comprising a chosen imprinting duration,
a duration for which the imprinting of the active effect is carried out being equal to the chosen imprinting duration.

20. The method as claimed in claim 2, wherein the constructing of the electrical imprinting signal based on the digital signature of the reference product comprises:
obtaining a first electrical signal by applying an inverse Fourier transform to the at least one digital signature of the reference product.
